(19) United States
(12) Patent Application Publication
(10) Pub. No.: US 2020/0000485 A1
(43) Pub. Date: Jan. 2, 2020

(54) CONSTRICTION REMOVAL METHOD

(71) Applicant: OLYMPUS CORPORATION, Tokyo (JP)

(72) Inventor: Kosuke MOTAI, Saitama (JP)

(73) Assignee: OLYMPUS CORPORATION, Tokyo (JP)

(21) Appl. No.: 16/025,115

(22) Filed: Jul. 2, 2018

(51) Int. Cl. A61B 17/22; A61B 18/14

(52) U.S. Cl. CPC A61B 17/22031; A61B 2018/00422; A61B 18/1492

(57) ABSTRACT

A constriction removal method using a high-frequency knife (8) and a collecting instrument (7) having a longitudinal axis and an increasing-diameter part (12), whose diameter can be increased, at the distal end, the constriction removal method including: inserting the collecting instrument (7) from a proximal end opening of a constricted lumen of a constricted portion; allowing the increasing-diameter part (12) to pass through the constricted lumen and to project from the distal-end opening of the constricted lumen and allowing the diameter of the increasing-diameter part (12) having passed through the constricted lumen to increase toward the radially outer side of the constricted lumen; hooking ends of the increasing-diameter part (12) having passed through the constricted lumen on an edge of the distal-end opening of the constricted lumen; positioning the high-frequency knife (8) on the radially outer side of the proximal end opening of the constricted lumen; cylindrically coring out the constricted lumen with the high-frequency knife (8) in a state in which the ends of the increasing-diameter part (12) are hooked on the edge of the distal-end opening of the constricted lumen; and collecting the cored-out constricted lumen by pulling the collecting instrument (7) toward the proximal end of the collecting instrument (7).

### TECHNICAL FIELD

The present invention relates to a constriction removal method.

### BACKGROUND ART

In a conventional method for removing a constriction formed in a lumen, such as the esophagus, the diameter of wires arranged at intervals in the circumferential direction on the outer circumference of a sheath inserted into the center of the constriction is increased while a high-frequency current is supplied to the wires, and subsequently, the sheath is rotated about the longitudinal axis while the high-frequency current is supplied to the wires (for example, see Patent Literature 1).

Specifically, a constriction formed in a lumen is removed according to the process including steps 1 to 3 below.

Step 1: A sheath is inserted into the center opening of the constriction in the longitudinal-axis direction of the lumen, and the sheath is moved in the longitudinal direction to align the wires with the constriction.

Step 2: The diameter of the wires is increased while a high-frequency current is supplied to the wires to cut the constricted portion radially outward with the wires.

Step 3: The sheath is rotated about the longitudinal axis while the high-frequency current is supplied to the wires to incise the constricted portion in the circumferential direction with the wires.

### CITATION LIST

### Patent Literature

### {PTL 1} Japanese Unexamined Patent Application Publication No. 2008-295729

### SUMMARY OF INVENTION

An aspect of the present invention is a constriction removal method using a high-frequency knife and a collecting instrument having a longitudinal axis and an increasing-diameter part, whose diameter can be increased, at the distal end, the constriction removal method including: inserting the collecting instrument from a proximal end opening of a constricted lumen of a constricted portion; allowing the increasing-diameter part to pass through the constricted lumen and to project from the distal-end opening of the constricted lumen and allowing the diameter of the increasing-diameter part having passed through the constricted lumen to increase toward the radially outer side of the constricted lumen; hooking ends of the increasing-diameter part having passed through the constricted lumen on an edge of the distal-end opening of the constricted lumen; positioning the high-frequency knife on the radially outer side of the proximal end opening of the constricted lumen; cylindrically coring out the constricted lumen with the high-frequency knife in a state in which the ends of the increasing-diameter part are hooked on the edge of the distal-end opening of the constricted lumen; and collecting a cored-out tissue piece of the constricted lumen by pulling the collecting instrument toward the proximal end of the collecting instrument.

### BRIEF DESCRIPTION OF DRAWINGS

FIG. 1 is a perspective view of a distal end portion of a constriction removal system used in a constriction removal method according to an embodiment of the present invention.

FIG. 2 is a vertical cross section showing an over tube provided in the constriction removal system in FIG. 1.

FIG. 3 is a side view showing a collecting instrument provided in the constriction removal system in FIG. 1.

FIG. 4 is a flowchart for explaining the constriction removal method in FIG. 1.

FIG. 5 is a partial vertical cross section showing a state in which the over tube is inserted to a position near a constricted portion in a lumen, in the constriction removal method in FIG. 4.

FIG. 6 is a partial vertical cross section showing a state in which a guide wire is inserted through the opening of the constricted portion, in the constriction removal method in FIG. 4.

FIG. 7 is a partial vertical cross section for explaining a task of pushing a tapered portion at the distal end of the collecting instrument into the opening of the constricted portion, in the constriction removal method in FIG. 4.

FIG. 8 is a partial vertical cross section showing a state in which a flap of the collecting instrument has penetrated through the opening of the constricted portion, in the constriction removal method in FIG. 4.

FIG. 9 is a partial vertical cross section showing a state in which the penetrated collecting instrument in FIG. 8 is pulled toward the proximal end side.

FIG. 10 is a partial vertical cross section showing a state in which tissue in the constricted portion is incised in the longitudinal-axis direction with a high-frequency knife, in a state in which the collecting instrument is pulled in FIG. 9.

FIG. 11 is a partial vertical cross section showing a state in which the tissue in the constricted portion is incised in the circumferential direction by rotating the over tube from the state in FIG. 10.

FIG. 12 is a partial vertical cross section showing a state in which incision of the constricted portion has been completed from the state in FIG. 11, and the tissue has been cylindrically cored out.

FIG. 13 is a partial vertical cross section showing a process of collecting a tissue piece that has been cylindrically cored out in FIG. 12.

FIG. 14 is a side view showing an example of the high-frequency knife used in the constriction removal system in FIG. 1.

FIG. 15 is a vertical cross section showing a modification of the over tube in FIG. 2.

FIG. 16 is a vertical cross section showing a modification of the collecting instrument in FIG. 3.

FIG. 17 is a partial vertical cross section for explaining a task of attaching an incision-surface protection sheet, which is performed after the cylindrical tissue piece has been collected in FIG. 12.

FIG. 18 is a partial vertical cross section showing a state in which the incision-surface protection sheet is attached to an incision surface of the constricted portion in the task explained in FIG. 17.

FIG. 19 is a partial perspective view showing a modification of the constriction removal system in FIG. 1.

FIG. 20 is a partial perspective view showing another modification of the constriction removal system in FIG. 1.

### DESCRIPTION OF EMBODIMENTS

A constriction removal method according to an embodiment of the present invention will be described below with reference to the drawings.

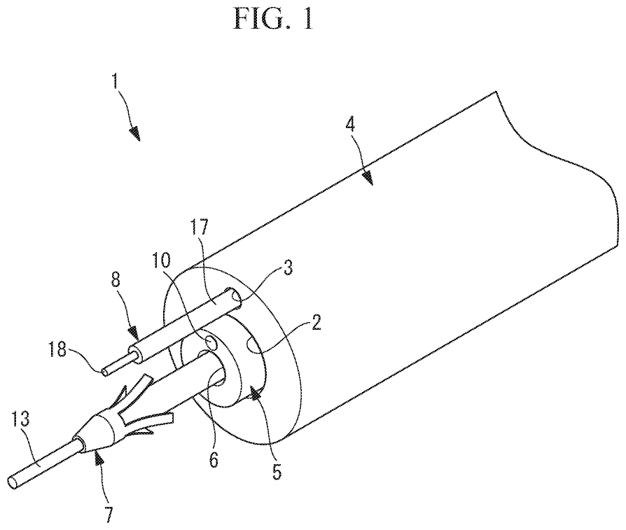

As shown in FIG. 1, a constriction removal system 1 used in the constriction removal method according to this embodiment includes: an over tube 4, which has a plurality of channels 2 and 3 penetrating in the longitudinal direction; an endoscope 5 inserted through the first channel 2 in the over tube 4; a collecting instrument 7 inserted through the channel 6 in the endoscope 5; and a high-frequency knife 8 inserted through the second channel 3 in the over tube 4.

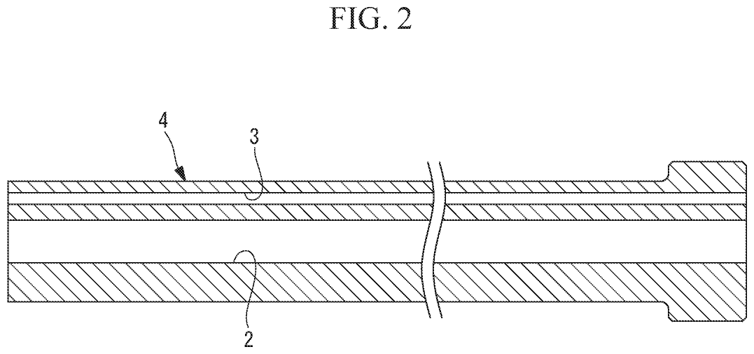

As shown in FIG. 2, the over tube 4 is a multi-channel tube that is made of a flexible material. The first channel 2 is formed at the center in cross section, and the second channel 3 is formed at a position radially shifted from the center in cross section. It is desirable that the over tube 4 to be employed be braided or be made of a material whose rigidity is adjusted to be relatively high so that a rotational force about the longitudinal axis applied at the proximal end is transmitted to the distal end.

The endoscope 5 includes an observation optical system 10 that is provided at the distal end face thereof and is used for observation in the forward direction, and a channel 6 that opens substantially in the center of the distal end face.

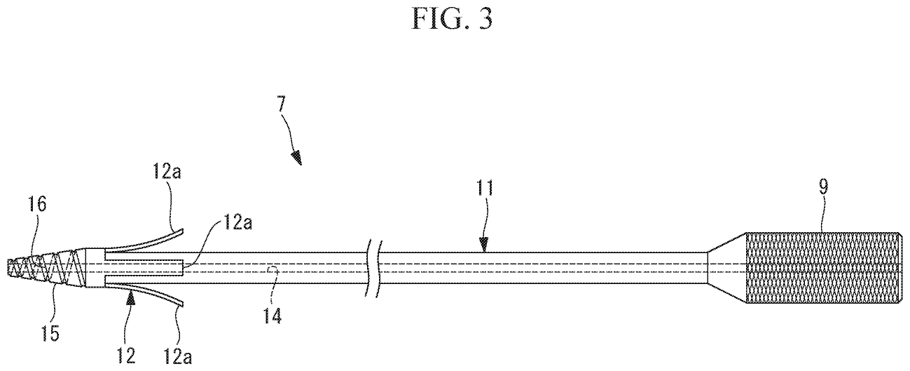

As shown in FIG. 3, the collecting instrument 7 includes a thin, long tubular body part 11 penetrating through the channel 6 in the endoscope 5 and projecting from the distal end face, a flap (increasing-diameter part) 12 provided at the distal end of the body part 11, and a through-hole 14 penetrating the body part 11 in the longitudinal direction and allowing the guide wire 13 to pass therethrough. Furthermore, a tapered portion 15, which is tapered toward the distal end, is provided at the distal end of the body part 11, and a spiral groove (projections and recesses) 16 is formed on the surface of the tapered portion 15. Denoted by reference sign 9 in FIG. 3 is a handle gripped by a user, and the outer circumferential surface of the handle 9 is, for example, knurled for increased friction.

The flap 12 includes a plurality of flap ends (ends of the increasing-diameter part) 12a arranged side-by-side in the circumferential direction. The flap 12 has a distal end and a proximal end, and the flap ends 12a constitute the proximal end of the flap 12. Furthermore, the flap ends 12a are formed of an elastic material and have a thin, long shape extending along the longitudinal axis of the body part 11. The distal end of the flap 12 is fixed to the outer surface of the distal end portion of the body part 11, and the flap ends 12a are curled such that the distance from the body part 11 gradually increases toward the proximal ends. Hence, the plurality of flap ends 12a have such a shape that they extend radially when viewed in the longitudinal-axis direction of the body part 11.

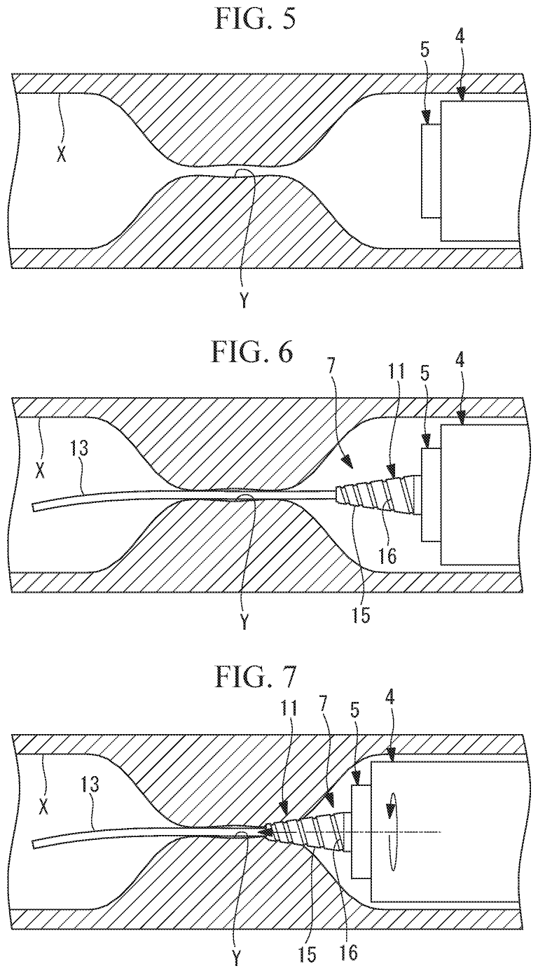

When the flap ends 12a are elastically deformed to positions parallel to the outer surface of the body part 11, and the curl of the flap ends 12a is straightened, the flap ends 12a can be contracted until the outside diameter thereof is smaller than the inside diameter of the channel 6. The flap ends 12a are shaped so as to be curled and increased in diameter until they cannot pass through a constricted lumen at the center of a constricted portion Y in a free state in which no external force is applied (see FIG. 5).

When the flap ends 12a pass through the channel 6 in the endoscope 5 and the constricted lumen in the constricted portion Y, the flap ends 12a are contracted and pass therethrough easily. Once the flap ends 12a have passed through the constricted lumen in the constricted portion Y, the flap ends 12a become free and expand radially.

As shown in FIG. 1, the high-frequency knife 8 includes an electrode 18 exposed at the distal end of a elongated sheath 17. When the high-frequency knife 8 is projected from the distal end face of the over tube 4 through the second channel 3, the electrode 18 is disposed at a position radially shifted from the longitudinal axis of the over tube 4 so as to be substantially parallel to the longitudinal axis. By moving the high-frequency knife 8 in the longitudinal-axis direction through the second channel 3, it is possible to move the electrode 18 forward or backward.

A constriction removal method using the thus-configured constriction removal system 1 will be described below.

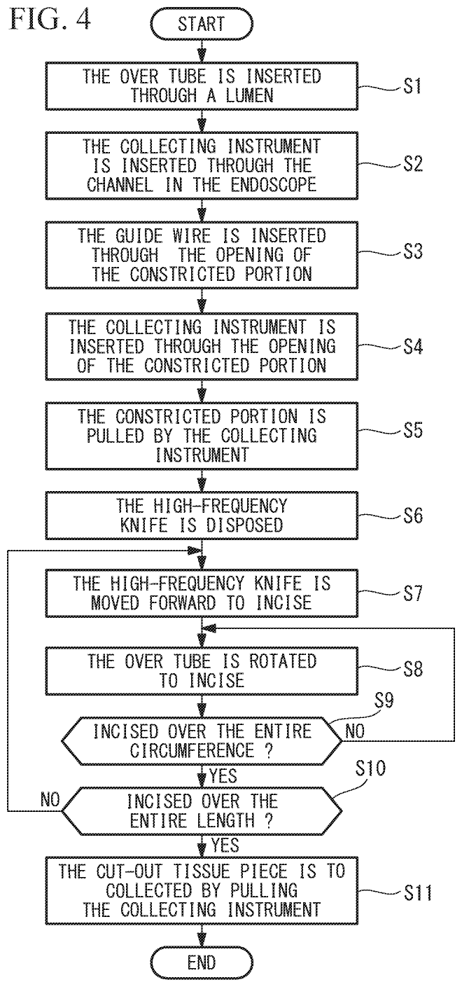

In the constriction removal method according to this embodiment, as shown in FIG. 4, the over tube 4 having the endoscope 5 inserted through the first channel 2 is inserted through a lumen X, the over tube 4 is moved forward while the state inside the lumen X is observed by using the endoscope 5, and the distal end face of the over tube 4 is made to face the constricted portion Y, as shown in FIG. 5 (step S1).

The collecting instrument 7 is inserted through the channel 6 in the endoscope 5 (step S2), and the guide wire 13 inserted through the through-hole 14 in the body part 11 is projected from the distal end of the body part 11. Then, while an image obtained by the endoscope 5 is viewed, the projected guide wire 13 is inserted from the proximal end opening of the constricted lumen at the center of the constricted portion Y, as shown in FIG. 6 (step S3).

Next, the body part 11 of the collecting instrument 7 is moved forward by using the guide wire 13 as a guide, and the body part 11 is pushed into the constricted lumen in the constricted portion Y (step S4). At this time, as shown in FIG. 7, while the tapered portion 15 provided at the distal end of the body part 11 is pushed into the constricted lumen, the body part 11 is rotated about the longitudinal axis. By doing so, the spiral groove 16 formed in the tapered portion 15 bites into the tissue in the constricted portion Y. By doing so, it is possible to obtain a thrust for moving the body part 11 forward by the rotation of the body part 11, and thus, it is possible to easily insert the collecting instrument 7 through the narrow constricted lumen in the constricted portion Y and make it pass through the constricted lumen.

In the process of inserting the collecting instrument 7 into the constricted lumen in the constricted portion Y, the flap 12 provided on the collecting instrument 7 can easily pass through the constricted lumen because the flap 12 is pushed radially inward and is contracted by the constricted portion Y.

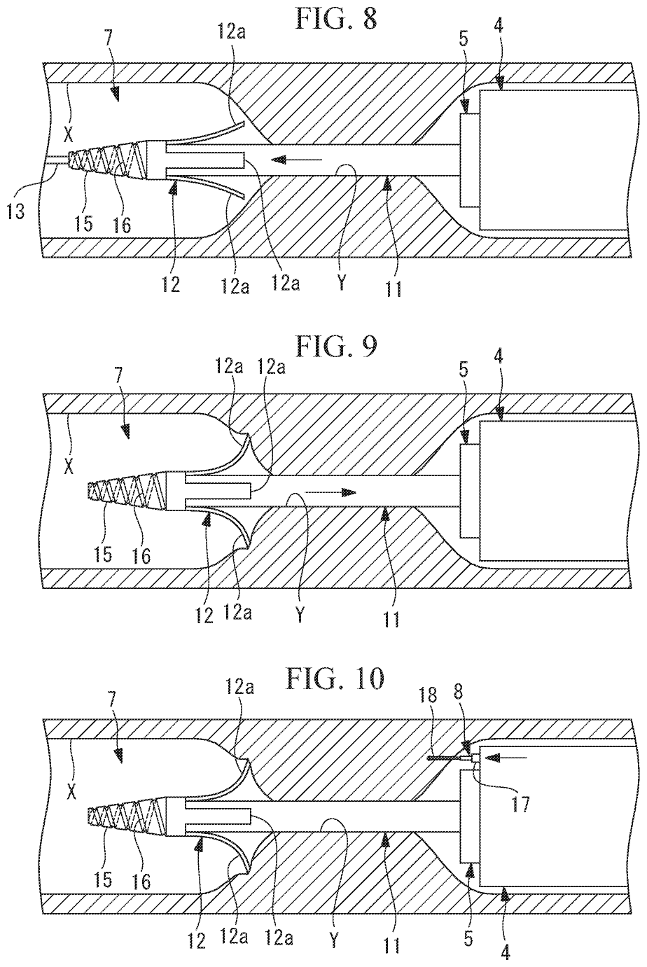

As shown in FIG. 8, when the flap 12 has passed through the constricted lumen and is projected from the distal-end opening of the constricted lumen, the diameter of the flap 12 radially increases due to the elastic restoring force thereof. In this state, by pulling the constriction toward the proximal end using the collecting instrument 7, as shown in FIG. 9, the flap ends 12a of the flap 12 are pressed against the distal side of the constricted portion Y. In other words, the flap ends 12a of the flap 12 having passed through the constricted lumen are hooked on the edge of the distal-end opening of the constricted lumen (step S5). Being hooked on the edge of the distal-end opening of the constricted lumen in the constricted portion Y, the flap 12 further increases in diameter. The guide wire 13 is removed at this time.

Thereafter, the high-frequency knife 8 is inserted through the second channel 3 in the over tube 4 and, as shown in FIG. 10, the electrode 18 of the high-frequency knife 8 is projected to the front side from the distal end face of the over tube 4 (step S6), and the high-frequency knife 8 is disposed on the radially outer side of the proximal end opening of the constricted lumen in the constricted portion Y. Then, in a state in which a high-frequency current is supplied to the electrode 18, the high-frequency knife 8 is moved forward to cut the constricted portion Y from the proximal side in the length direction of the lumen X (step S7). Because the constricted lumen in the constricted portion Y is incised with the high-frequency knife 8 from the proximal end side of the high-frequency knife 8 with the flap ends 12a being hooked on the edge of the distal-end opening of the constricted lumen in the constricted portion Y by the collecting instrument 7, it is possible to hold the tissue of the constricted lumen in the constricted portion Y so as not to escape from the high-frequency knife 8, and thus, to perform stable incision.

Next, in a state in which the length of the cut in the constricted portion Y is smaller than or equal to the length of the electrode 18 exposed from the distal end of the sheath 17, and while the high-frequency current continues to be supplied to the electrode 18 of the high-frequency knife 8, a rotational force about the endoscope 5 is applied to the proximal end of the over tube 4. By doing so, as shown in FIG. 11, the over tube 4 is rotated about the longitudinal axis to incise the constricted portion Y with the high-frequency knife 8 in the circumferential direction (step S8).

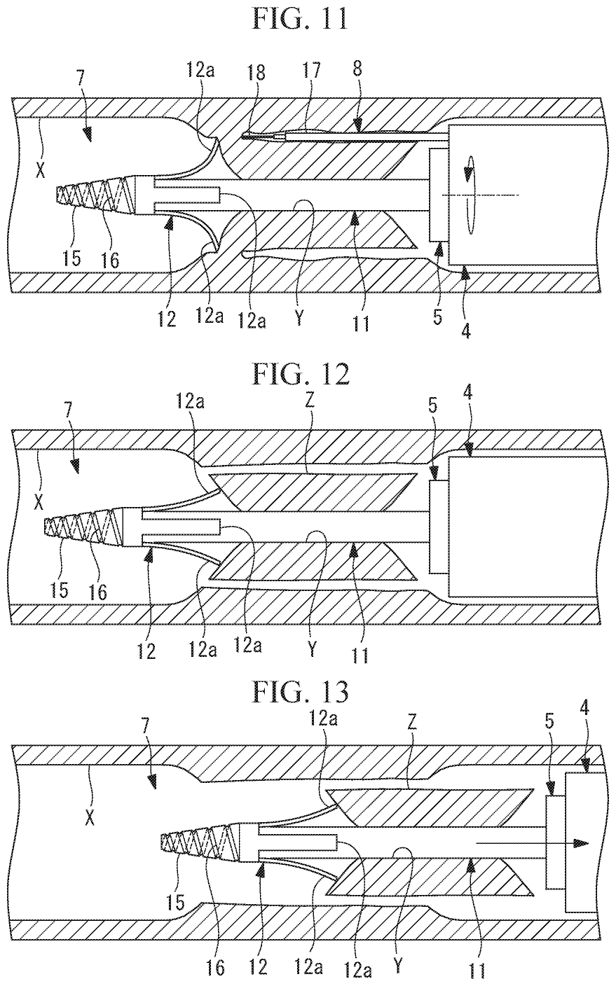

It is determined whether or not the constricted portion Y has been incised over the entire circumference in the circumferential direction (step S9), and, if the constricted portion Y has not been incised over the entire circumference, the process is repeated from step S8. If the constricted portion Y has been incised over the entire circumference, it is determined whether or not the constricted portion Y has been incised over the entire length (step S10). If the constricted portion Y has not been incised over the entire length, the process is repeated from step S7. Then, as shown in FIG. 12, when the constricted portion Y has been incised over the entire length and over the entire circumference with the high-frequency knife 8, it is possible to core out a cylindrical tissue piece Z from the constricted lumen in the constricted portion Y.

Once the constricted portion Y has been cored out, the force applied to the flap ends 12a from the distal side of the constricted portion Y is lifted, allowing the flap ends 12a to slightly contract due to the elastic restoring force. Specifically, after the incision with the high-frequency knife 8, the flap ends 12a contract to a size smaller than the inside diameter of the hole formed in the constricted portion Y. Because the flap ends 12a are hooked on the distal side of the cored-out, cylindrical tissue piece Z of the constricted lumen in the constricted portion Y, as shown in FIG. 13, by pulling the collecting instrument 7 toward the proximal end, together with the over tube 4, it is possible to collect the cut-out tissue piece Z outside the body without leaving it in the lumen X (step S11).

In a conventional method in which a tissue piece Z is incised in the radial direction with wires to which a high-frequency current is supplied and is then cut out by rotating the wires in the circumferential direction, the cut-out tissue piece Z is divided into a plurality of pieces, and thus, it is difficult to collect the tissue piece Z without leaving it in the lumen X. In contrast, the constriction removal method according to this embodiment has an advantage in that, because the constricted lumen in the constricted portion Y is cylindrically cored out with the body part 11 of the collecting instrument 7 penetrating therethrough and is hooked by the flap ends 12a, it is possible to more reliably collect the tissue piece Z outside the body without leaving it in the lumen X.

There is another advantage in that, because the over tube 4 disposed outside the endoscope 5 is rotated in a state in which the endoscope 5 inserted through the first channel 2 is fixed, the field of view of the endoscope 5 does not rotate.

In this embodiment, the techniques below can be employed.

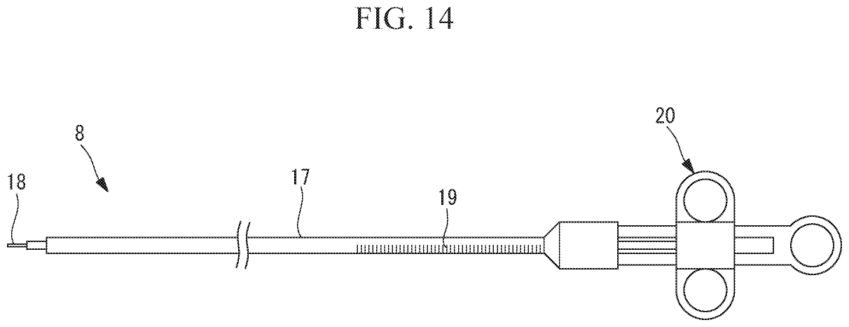

First, to check the amount of movement of the high-frequency knife 8 when the constricted portion Y is incised with the electrode 18 of the high-frequency knife 8, graduation marks 19 may be provided on the outer circumferential surface of the sheath 17 of the high-frequency knife 8 exposed outside the body from the proximal end of the over tube 4. As shown in FIG. 14, the high-frequency knife 8 includes, at the proximal end of the sheath 17, a slider 20 that advances and retracts the electrode 18 relative to the sheath 17 in the longitudinal-axis direction, and the graduation marks 19 are provided on the outer circumferential surface of the sheath 17 on the distal-end side of the slider 20.

Second, although it has been described that it is desirable that the over tube 4 to be employed be braided or be one whose rigidity is adjusted so that a rotational force about the longitudinal axis applied at the proximal end is transmitted to the distal end, in addition to this, it is desirable that the over tube 4 be flexible enough to passively bend in accordance with the bending action of the endoscope 5 inserted through the first channel 2.

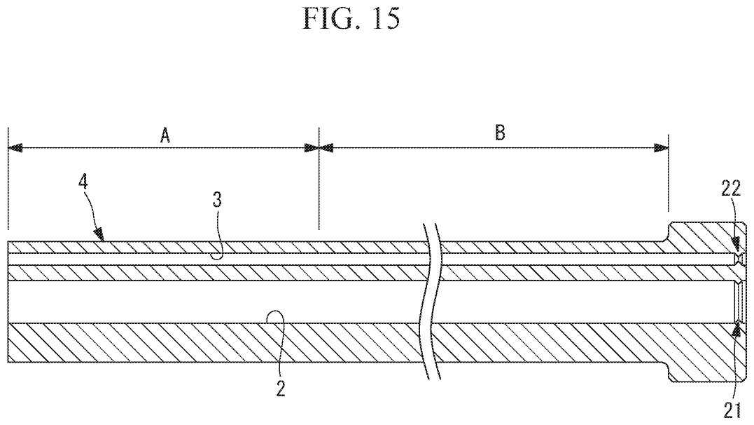

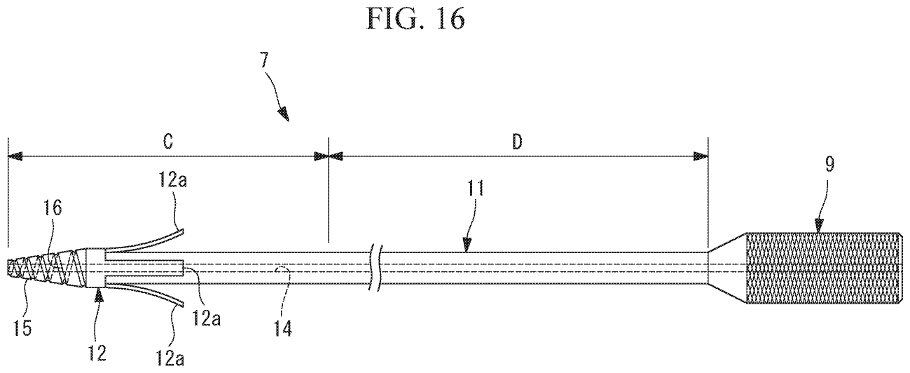

From this stand point, as shown in FIG. 15, it is possible to configure the system such that only a distal end portion A corresponding to a bending section of the endoscope 5 inserted through the first channel 2 has high flexibility, and the other area B is braided or is made of a rigid material so that it can effectively transmit the rotational force.

Third, as shown in FIG. 15, it is desirable that the channels 2 and 3 in the over tube 4 respectively have valves 21 and 22 that project radially inward and seal between the inner surface of the first channel 2 and the outer circumferential surface of the endoscope 5 and between the inner surface of the second channel 3 and the outer circumferential surface of the sheath 17 of the high-frequency knife 8. It is desirable that the valve 21 in the first channel 2 have low friction with respect to the endoscope 5 to allow the endoscope 5 to easily advance and retract, while maintaining airtightness. It is desirable that the valve 22 in the second channel 3 have relatively high friction with respect to the high-frequency knife 8 inserted through the second channel 3 to allow the high-frequency knife 8 to be fixed with respect to the over tube 4 in the longitudinal-axis direction, while maintaining airtightness.

Fourth, it is possible to configure the system such that a balloon (not shown) that fills the gap with respect to the lumen X when expanded is provided near the distal end portion of the over tube 4, and incision with the high-frequency knife 8 is performed after the balloon is expanded. This configuration makes it possible to center the over tube 4 with respect to the lumen X and, thus, to stably incise and cylindrically core out the constricted portion Y.

Fifth, although it has been described that the channel 6 in the endoscope 5 is located substantially in the center of the endoscope 5 in cross section and that the first channel 2 in the over tube 4 is located at the center of the over tube 4 in cross section, they may be shifted from the centers.

Sixth, as shown in FIG. 16, it is desirable that, in the body part 11 of the collecting instrument 7, a first area C, which is inserted through the constricted lumen in the constricted portion Y, have a higher rigidity than a second area D, which is the area other than the first area C. This configuration minimizes bending of an area of the body part 11 serving as the axis during rotation of the high-frequency knife 8 and thus makes it possible to stably rotate the over tube 4. Because increasing the length of the relatively rigid first area C makes the bending operation of the endoscope 5 difficult, it is desirable that the system be set up so as to satisfy a good balance between rotation stability and ease of bending.

It is possible to configure the system such that a braid (not shown) that is made of a metal mesh body is provided on the outer circumferential surface of the body part 11 to stiffen the first area C, and the braid is grounded. This configuration makes it possible to use the braid as a return electrode for collecting a high-frequency current supplied to the electrode 18 of the high-frequency knife 8, and thus, it is possible to increase the current density of the high-frequency current flowing through the constricted portion Y to enable efficient incision.

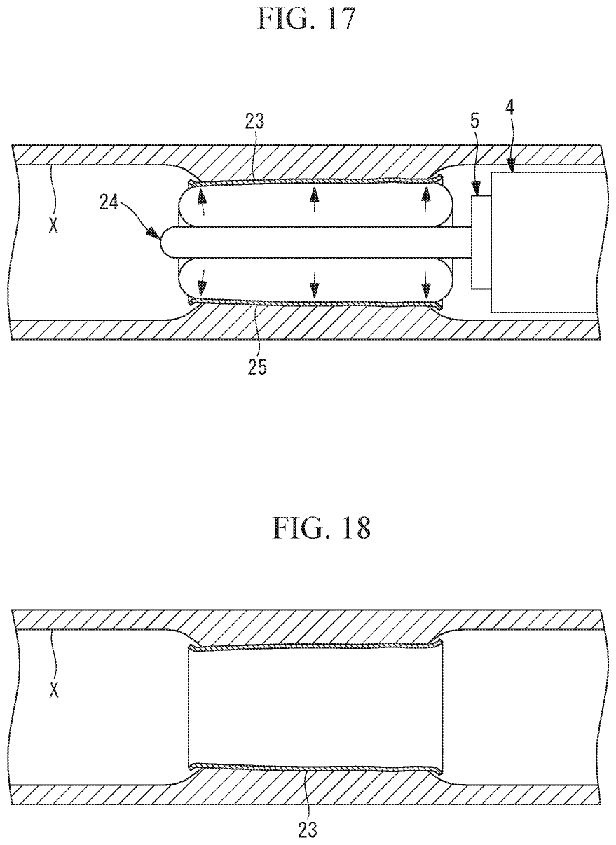

Seventh, it is possible to configure the system such that, after the cored-out tissue piece Z is collected outside the body by using the collecting instrument 7, as shown in FIGS. 17 and 18, an incision-surface protection sheet 23, such as a cell sheet, rolled in a cylindrical shape is positioned on an incision surface, a balloon 25 of a balloon catheter 24 disposed inside the incision-surface protection sheet 23 is inflated, and the spread-out incision-surface protection sheet 23 is applied to the overall incision surface. This configuration makes it possible to prevent formation of scar tissue on the incision surface, and consequently, a recurrence of constriction.

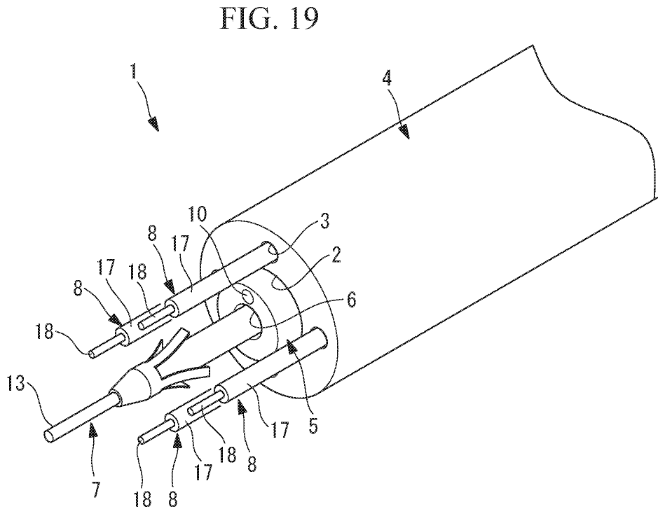

Eighth, although it was described that the constricted portion Y is cylindrically cored out by rotating a single high-frequency knife 8 by 360°, instead, as shown in FIG. 19, it is also possible to configure the system such that a plurality of second channels 3 are provided at positions equal distances away from the rotation center of the over tube 4 in the radial direction to incise the constricted portion Y with a plurality of high-frequency knives 8 projected from the second channels 3. For example, when incision is to be made by using four high-frequency knives 8, the over tube 4 needs to be rotated only by 90°. Thus, it is possible to reduce the time needed for incision.

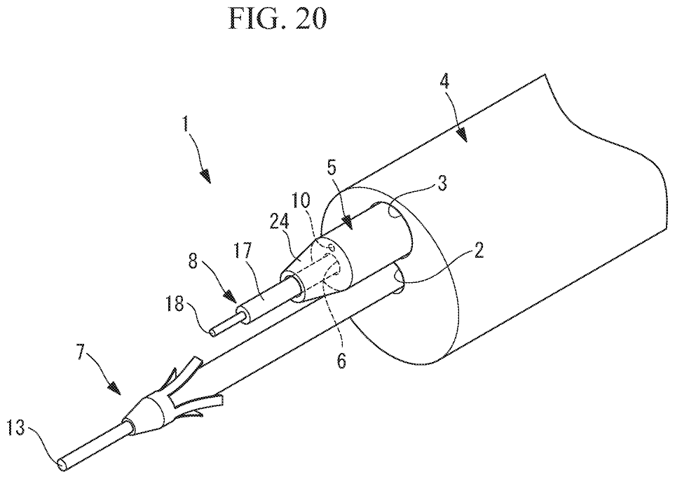

Ninth, although it was described that the endoscope 5 and the collecting instrument 7 are inserted through the first channel 2, and the high-frequency knife 8 is inserted through the second channel 3, instead, as shown in FIG. 20, it is also possible to configure the system such that the collecting instrument 7 is inserted through the first channel 2, and the endoscope 5 is inserted through the second channel 3, and the high-frequency knife 8 is disposed via the channel 6 in the endoscope 5.

This configuration makes it possible to perform incision while always monitoring the status of incision with the high-frequency knife 8 by using the endoscope 5. In this case, as shown in FIG. 20, the distal end of the endoscope 5 may be covered with a clear cap 26 to prevent the observation optical system 10 of the endoscope 5 from being stained with blood or the like produced by incision of the constricted portion Y.

Tenth, although the flap 12 whose diameter increases by the elastic restoring force thereof has been described as an example of the increasing-diameter part, instead, it is possible to employ a balloon whose diameter increases by supplying fluid thereto.

Examples of the lumen X in which a constriction to be removed by the constriction removal method according to this embodiment include: the alimentary canal including the esophagus, the cardiac region of the stomach, the pyloric region of the stomach, the duodenum, the small intestine, the colon, and the rectum; and lumens other than the alimentary canal.

### REFERENCE SIGNS LIST

2 first channel (channel)
3 second channel (channel)
4 over tube
7 collecting instrument
8 high-frequency knife
12 flap (increasing-diameter part)
12a flap end (end of increasing-diameter part)
Y constricted portion
Z tissue piece

1. A constriction removal method using a high-frequency knife and a collecting instrument having a longitudinal axis and an increasing-diameter part, whose diameter can be increased, at the distal end, the constriction removal method comprising:
inserting the collecting instrument from a proximal end opening of a constricted lumen of a constricted portion;
allowing the increasing-diameter part to pass through the constricted lumen and to project from the distal-end opening of the constricted lumen and allowing the diameter of the increasing-diameter part having passed through the constricted lumen to increase toward the radially outer side of the constricted lumen;
hooking ends of the increasing-diameter part having passed through the constricted lumen on an edge of the distal-end opening of the constricted lumen;
positioning the high-frequency knife on the radially outer side of the proximal end opening of the constricted lumen;
cylindrically coring out the constricted lumen with the high-frequency knife in a state in which the ends of the increasing-diameter part are hooked on the edge of the distal-end opening of the constricted lumen; and
collecting a cored-out tissue piece of the constricted lumen by pulling the collecting instrument toward the proximal end of the collecting instrument.

2. The constriction removal method according to claim 1, wherein the constricted lumen is cylindrically cored out with the high-frequency knife by moving the high-frequency knife in the longitudinal-axis direction of the collecting instrument and then in the circumferential direction about the longitudinal axis while a high-frequency current is supplied to the high-frequency knife.

3. The constriction removal method according to claim 2, wherein the constricted lumen is cylindrically cored out by alternately repeating incising the constricted lumen in the longitudinal-axis direction by moving the high-frequency knife in the longitudinal-axis direction and incising the constricted lumen in the circumferential direction by moving the high-frequency knife in the circumferential direction.

4. The constriction removal method according to claim 2, wherein the high-frequency knife is moved in the circumferential direction by rotating an over tube about the longitudinal axis of the collecting instrument, the over tube having a plurality of channels penetrating in the longitudinal direction, the collecting instrument being inserted through the first channel, and the high-frequency knife being inserted through a second channel.
